(19) United States
(12) Patent Application Publication
(10) Pub. No.: US 2017/0340836 A1
(43) Pub. Date: Nov. 30, 2017

(54) A Multi-State Ratchet Mechanism for Drug Injection Devices

(71) Applicant: Novo Nordisk A/S, Bagsvaerd (DK)

(72) Inventor: Ebbe Kiilerich, Copenhagen NV (DK)

(21) Appl. No.: 15/531,953

(22) PCT Filed: Dec. 22, 2015
(86) PCT No.: PCT/EP2015/080962

§ 371 Date: May 31, 2017

(30) Foreign Application Priority Data
Dec. 29, 2014 (EP) 14200379.7

(51) Int. Cl. A61M 5/315

(52) U.S. Cl. CPC A61M 5/31585; A61M 5/31553

(57) ABSTRACT

The present invention relates in a first aspect to a multi-state ratchet mechanism for a drug injection device. The multi-state ratchet mechanism comprises a ratchet gear member and a cooperating ratchet pawl member. An annular inner surface of the ratchet gear member comprises a first ring shaped surface portion with a first predetermined cross-sectional profile comprising a first asymmetrical toothing arranged at a first axial position of the inner annular surface. The annular inner surface of the ratchet gear member further comprises a second ring shaped surface portion arranged at a second axial position of the inner surface axially spaced apart from the first axial position. The second ring shaped surface portion comprises a second predetermined cross-sectional profile differing from the first predetermined cross-sectional profile. The ratchet pawl member is axially displaceable between the first ring shaped surface portion and the second ring shaped surface portion to provide first and second operating states, respectively, of the multi-state ratchet mechanism. The first operational state of the multi-state ratchet mechanism provides unidirectional rotation between the ratchet gear member and the ratchet pawl member in a first direction. The present multi-state ratchet mechanism is particularly suitable for drug injection devices such as user operated drug injection devices for administration of insulin for treating diabetes.

The present invention relates in a first aspect to a multi-state ratchet mechanism for a drug injection device. The multi-state ratchet mechanism comprises a ratchet gear member and a cooperating ratchet pawl member. An annular inner surface of the ratchet gear member comprises a first ring shaped surface portion with a first predetermined cross-sectional profile comprising a first asymmetrical toothing arranged at a first axial position of the inner annular surface. The annular inner surface of the ratchet gear member further comprises a second ring shaped surface portion arranged at a second axial position of the inner surface axially spaced apart from the first axial position. The second ring shaped surface portion comprises a second predetermined cross-sectional profile differing from the first predetermined cross-sectional profile. The ratchet pawl member is axially displaceable between the first ring shaped surface portion and the second ring shaped surface portion to provide first and second operating states, respectively, of the multi-state ratchet mechanism. The first operational state of the multi-state ratchet mechanism provides unidirectional rotation between the ratchet gear member and the ratchet pawl member in a first direction. The present multi-state ratchet mechanism is particularly suitable for drug injection devices such as user operated drug injection devices for administration of insulin for treating diabetes.

### BACKGROUND OF THE INVENTION

The present invention relates to a multi-state ratchet mechanism useful in numerous types of drug injection devices. Ratchet mechanisms are well-known in drug administration devices in order to only allow movement or rotation between a first member and a second member in one direction. To lock movement or rotation in both directions between the first and second members in connection with the use of such prior art ratchet mechanisms or to allow movement or rotation in the normally locked direction, a second clutch mechanism is normally needed. This second clutch mechanism could either lock the first and second members, engage a second, and reversely oriented, ratchet while decoupling the first ratchet, or switch to an indexing (click) mechanism. Such a clutch mechanism will require extra parts and/or more complicated parts to increase manufacturing costs and size of the ratchet mechanism. A clutch mechanism may introduce various synchronisation problems in connection with switching between the different states of the ratchet mechanism. The clutch mechanism may for example introduce tooth jumping when such a clutch based ratchet mechanism is under load and switches between a locked state and an unlock state. In the following disclosure of the present invention, aspects and embodiments will be described which address one or more of the above objects or which address objects apparent from the disclosure as well as from the description of exemplary embodiments.

### SUMMARY OF INVENTION

A first aspect of the invention relates to a multi-state ratchet mechanism for a drug injection device. The multi-state ratchet mechanism comprises a ratchet gear member comprising an outer surface of predetermined shape and dimensions and an inner annular surface. The inner annular surface comprises a first ring shaped surface portion with a first predetermined cross-sectional profile comprising a first asymmetrical toothing arranged at a first axial position of the inner annular surface where each asymmetrical tooth of the first asymmetrical toothing comprises a first sloped edge and a second sloped edge wherein a slope of the first sloped edge is steeper than a slope of the second sloped edge. The inner annular surface further comprises a second ring shaped surface portion arranged at a second axial position of the inner surface axially spaced apart from the first axial position and having a second predetermined cross-sectional profile differing from the first predetermined cross-sectional profile. A ratchet pawl member of the multi-state ratchet mechanism is axially displaceable between the first ring shaped surface portion and the second ring shaped surface portion to provide first and second operating states, respectively, of the multi-state ratchet mechanism. The ratchet pawl member comprising a pawl tooth or finger comprising a forward facing radially projecting stop surface configured for engagement with the first sloped edge or the second sloped edge of each asymmetrical tooth and a backward facing radially projecting stop surface configured for engagement with the opposite one of the first and second sloped edges of each asymmetrical tooth to, in the first state of the multi-state ratchet mechanism, provide unidirectional rotation in a first direction between the ratchet gear member and the ratchet pawl member. The different cross-sectional profiles of the first and second ring shaped surface portions of the ratchet gear member provides multiple and different types of locked rotational states depending on the axial position of the ratchet pawl member relative to the ratchet gear member. Furthermore, the pawl tooth/finger of the ratchet pawl member does not need to disengage the first or second ring shaped surface portions of the ratchet gear member during switching between the different operational states of the multi-state ratchet mechanism. The ability to ensure constant engagement between the pawl tooth/finger and the ratchet gear member while shifting between different operational states is a significant advantage because it eliminates the previously discussed problems with tooth jumping during state switching. Finally, additional costs for manufacturing the present multi-state ratchet mechanism are low as the mechanism does not require the introduction of additional parts or components compared to prior art ratchet mechanisms.

In one embodiment of the multi-state ratchet mechanism, the second predetermined cross-sectional profile of the second ring shaped surface portion of the ratchet gear member comprises:
a substantially smooth surface physically engaging the pawl tooth or finger to, in the second state, provide bi-directional rotation between the ratchet gear member and the ratchet pawl member; or
a plurality of uniform sloped protrusions configured for engagement with the pawl tooth or finger to, in the second state, provide bi-directional rotation between the ratchet gear member and the ratchet pawl member.

The outer surface of the ratchet gear member may be cylindrical and the outer surface may form part of a pen shaped housing structure of a drug injection device in which the multi-state ratchet mechanism is integrated. The outer diameter of such a cylindrical ratchet gear member may be smaller than 30 mm, or smaller than 25 mm to match typical outer housing dimensions of the pen shaped housing structure of the drug injection device.

In another embodiment of the multi-state ratchet mechanism, the second predetermined cross-sectional profile of the second ring shaped surface portion comprises a second asymmetrical toothing, orientated oppositely to the first asymmetrical toothing,
each asymmetrical tooth of the second asymmetrical toothing comprises a first sloped edge and a second sloped edge wherein a slope of the first sloped edge is steeper than a slope of the second sloped edge to, in the second state of the multi-state ratchet mechanism, provide unidirectional rotation in a second direction, oppositely to the first direction, between the ratchet gear member and the ratchet pawl member in the second state of the multi-state ratchet mechanism.

In one preferred embodiment, the second predetermined cross-sectional profile of the second ring shaped surface portion comprises a toothing with an unconventional cross-sectional profile with at least three sloped edges as discussed in additional detail below with reference to the appended drawings. In the latter embodiment, the second predetermined cross-sectional profile comprises a first sloped edge and a second sloped edge wherein a slope of the first sloped edge is steeper than a slope of the second sloped edge; and

a third sloped edge arranged in-between the first sloped edge and the second sloped edge, wherein a slope of the third sloped edge is steeper than the slope of the second sloped edge such that the first sloped edge is configured for engaging the forward facing radially projecting stop surfaces of the pawl tooth and the third sloped edge is configured for engaging the backwards facing radially projecting stop surface of the pawl tooth thereby providing bidirectional rotational locking between the ratchet gear member and the ratchet pawl member in the second state of the multi-state ratchet mechanism. The slope of the first sloped edge may be substantially identical to the slope of the third sloped edge in the second predetermined cross-sectional profile of the second ring shaped surface portion.

The inner surface of the annular rotation member may comprise two, three or even more individual ring shaped surface portions each with a specific cross-sectional profile to provide different operational states of the multi-state ratchet mechanism. In one embodiment, the annular rotation member comprises a third ring shaped surface portion having a third predetermined cross-sectional profile. The third ring shaped surface portion is arranged at a third axial position of the inner annular surface and the third predetermined cross-sectional profile preferably differs from each of the first and second predetermined cross-sectional profiles. The ratchet pawl member being axially displaceable to the third ring shaped surface portion in a third operational state of the multi-state ratchet mechanism.

The third ring shaped surface portion may in some embodiments be arranged in-between the first and second ring shaped surface portions of the ratchet gear member in the axial direction of the ratchet gear member as discussed in additional detail below with reference to the appended drawings.

The ratchet pawl member may have various shapes and dimensions mating to the shape and dimensions of the ratchet gear member. The ratchet gear member may comprise a substantially flat annular body portion having a substantially ring shaped outer surface. The substantially flat annular body portion may comprise a central through going teethed aperture for receipt of a mating member such as a mating piston rod. The piston rod may comprise a threaded surface engaging or locking onto the teeth of the teethed aperture coupling the piston rod and the ratchet pawl member to each other.

The substantially flat annular body portion of the ratchet pawl member may comprise an elongate curved through going aperture extending along a first portion of the substantially ring shaped outer surface to form a narrow curved wall segment; wherein the narrow curved wall segment comprises the pawl finger.

The skilled person will understand that the ratchet pawl member may comprise a single tooth or pawl finger or a plurality of pawl teeth or pawl fingers. The plurality of pawl fingers may be able to increase mechanical strength of the coupling between the piston rod and the ratchet pawl member.

Another aspect of the invention relates to a drug injection device comprising:

a hollowing elongate housing structure,

an injection structure arranged inside the hollow housing structure and comprising a threaded axially extending piston rod coupled to a movable piston,

wherein the threaded axially extending piston rod is configured to advance the piston a predetermined axial distance inside a drug cartridge to deliver a predetermined dose of drug,

a user operable dose dial coupled to a dose adjustment structure,

a multi-state ratchet mechanism according to any of the above described embodiments thereof. The ratchet pawl member of the multi-state ratchet mechanism is coupled to the threaded axially extending piston rod via a coupling member. The drug injection device further comprises an axially displaceable user actuable button coupled to the ratchet gear member for axially displacing the ratchet gear member relative to the ratchet pawl member between at least the first and second states of the multi-state ratchet mechanism.

Details of the drug injection device and the integration of the present multi-state ratchet mechanism therein are discussed in additional detail below with reference to the appended drawings.

### BRIEF DESCRIPTION OF THE DRAWINGS

Preferred embodiments of the invention will be described in additional detail in connection with the appended drawings, in which:

FIG. 1 shows simplified schematic views 10, 20, 30, 40 of a multi-state ratchet mechanism 100, suitable for use in a drug injection device or other types of injection devices, in a first state of the multi-state ratchet mechanism for illustrating operating principles of the invention,

FIG. 2 shows simplified schematic views 10, 20, 30, 40 of a multi-state ratchet mechanism 100, suitable for use in a drug injection device or other types of injection devices, in a second state of the multi-state ratchet mechanism for illustrating operating principles of the invention,

FIG. 3 shows simplified schematic views 10, 20, 30, 40 of a multi-state ratchet mechanism 100, suitable for use in a drug injection device or other types of injection devices, in a third state of the multi-state ratchet mechanism for illustrating operating principles of the invention,

FIGS. 4A), B) and C) are respective schematic perspective views of three different operational states of a multi-state ratchet mechanism in accordance with a first embodiment of the invention,

FIGS. 5A), B) and C) are respective schematic perspective views of three different operational states of a multi-state ratchet mechanism in accordance with a second embodiment of the invention; and

FIG. 6A) shows an axial cross-sectional view of a drug injection device in an inactive state and comprising a multi-state ratchet mechanism according to any of the first and second embodiments thereof,

FIG. 6B) shows a radial cross-sectional view of the drug injection device taken through the multi-state ratchet mechanism in the inactive state of the device,

FIG. 7A) shows a second axial cross-sectional view of the drug injection device in the inactive state,

FIG. 7B) shows an expanded partial view of the drug injection device in the inactive state,

FIG. 8A) shows an axial cross-sectional view of the drug injection device in an active state,

FIG. 8B) shows a radial cross-sectional view of the drug injection device taken through the multi-state ratchet mechanism in the active state of the device,

FIG. 9A) shows a third axial cross-sectional view of the drug injection device in the active state; and

FIG. 9B) shows an expanded partial view of the drug injection device in the active state.

### DETAILED DESCRIPTION OF PREFERRED EMBODIMENTS

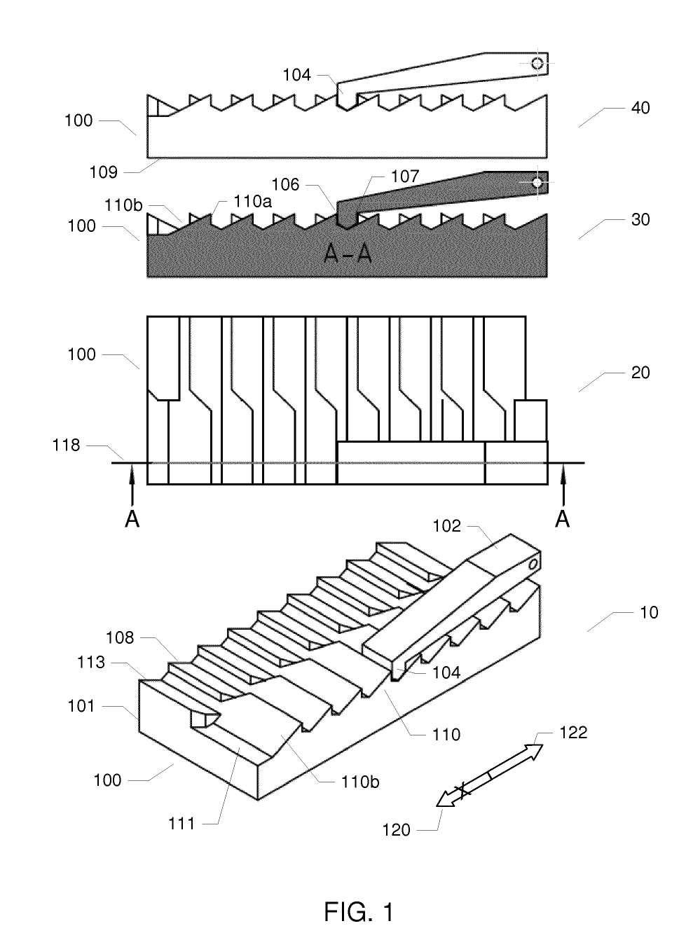

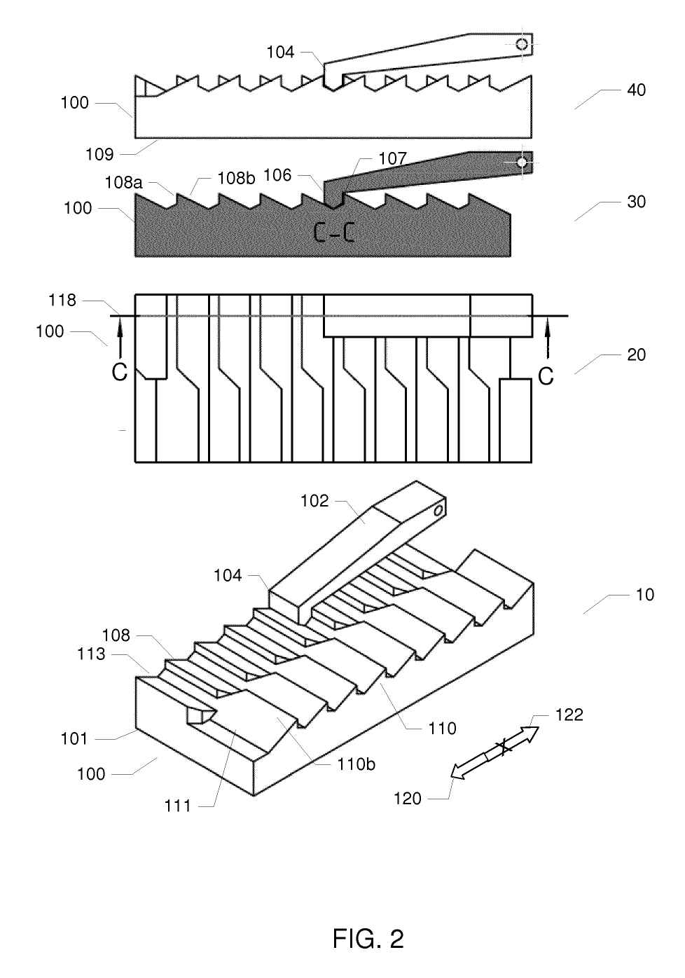

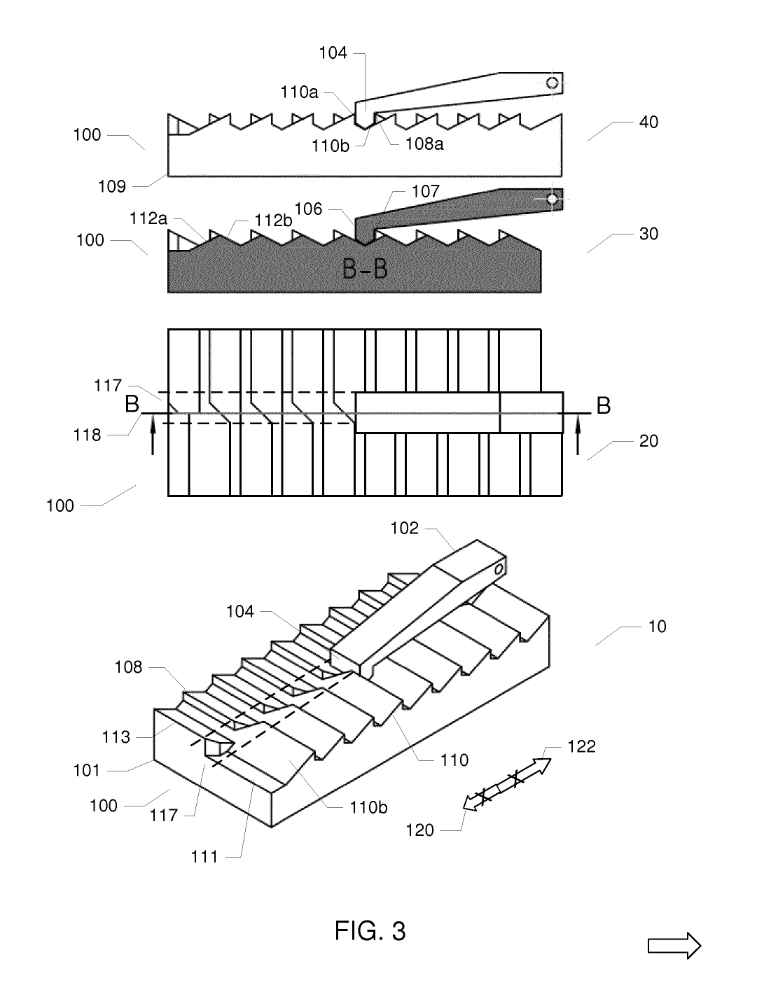

FIG. 1 shows simplified schematic views 10, 20, 30, 40 of a multi-state ratchet mechanism 100 suitable for use in a drug injection device or other types of injection devices. The multi-state ratchet mechanism 100 is placed in a first state of the multi-state ratchet mechanism and FIGS. 2 and 3 show second and third further states of the multi-state ratchet mechanism 100 as discussed below. The multi-state ratchet mechanism 100 comprises a ratchet gear member 101 and a cooperating ratchet pawl member 102 as illustrated in the top side oriented perspective view of the lowermost drawing 10. The view 20 is a top view of the multi-state ratchet mechanism 100 with an indication of a cross-sectional line 118. View 30 is a vertical cross-sectional view of the multi-state ratchet mechanism 100 through line 118 where teeth of a first asymmetrical toothing have been indicated by grey filling. View 40 is a side view of the multistate ratchet mechanism 100 illustrating teeth of the first asymmetrical toothing and teeth of a second asymmetrical toothing pointing in opposite direction to the first asymmetrical toothing as discussed in further detail below. A finger 104 or tooth of the ratchet pawl member 102 is biased against the first asymmetrical toothing to engage the latter.

The ratchet gear member 101 comprises an outer surface 109 of predetermined shape and dimensions, as most clearly depicted in view 40, which in practice may be a cylindrical wall section of a user operable button or a housing exterior of the drug injection device (not shown) in which the multi-state ratchet mechanism 100 is integrated. The outer surface 109 may therefore have a cylindrical shape for example comprising a corrugated surface to improve user manipulation and actuation. The ratchet gear member 101 comprises an inner surface 111, located oppositely to the outer surface 109, which comprises a first surface portion 110 having the first asymmetrical toothing arranged at a first position of the inner surface 111. The first asymmetrical toothing comprises a plurality of asymmetrical teeth having a first predetermined cross-sectional profile. Each asymmetrical tooth of the first asymmetrical toothing comprises a first sloped edge 110a and a second sloped edge 110b wherein a slope of the first sloped edge 110a is steeper than a slope of the second sloped edge 110b as best illustrated on view 30 showing the cross-sectional profile of the first asymmetrical toothing. The illustrated asymmetrical toothing is often referred to as a one-way toothing.

The inner surface 111 of the ratchet gear member 101 further comprises a second surface portion 113 arranged at a second position of the inner surface 111 spaced apart from the first position of the ratchet gear member 101 and having a second predetermined cross-sectional profile differing from the first predetermined cross-sectional profile. In the illustrated example, the second surface portion 113 also comprises an asymmetrical toothing which comprises a plurality of asymmetrical teeth having a second predetermined cross-sectional profile. As illustrated for example by tooth 108 of the second asymmetrical toothing, each tooth comprises a first sloped edge and a second sloped edge wherein a slope of the first sloped edge is steeper than a slope of the second sloped edge as best illustrated on view 10. However, the relative slopes of the first and second edges are reversed compared to the slopes of the sloped edges of the first asymmetrical toothing of the first surface portion 110. In this manner, the one-way toothing formed on the second surface portion 113 is oriented oppositely to the one-way toothing of the first surface portion 110.

The multi-state ratchet mechanism 100 comprises the above-mentioned ratchet pawl member 102 which is displaceable between the first and second surface portions 110, 113 of the inner surface 111 of the ratchet gear member 101. When the ratchet pawl member 101 is positioned at the first surface portion 110, the pawl finger or pawl tooth 104 of the pawl member 102 is biased against the first asymmetrical toothing such that forward and backward oriented stop surfaces of the pawl finger 104 engage or contact each tooth. The forward oriented stop surface 106 of the pawl finger 104 is best illustrated on view 30 where the backward oriented stop surface 107 of the pawl finger 104 likewise is indicated. Consequently, the one-way or asymmetrical toothing of the first surface portion 110 provides in cooperation with the ratchet pawl member 101 unidirectional relative motion, e.g. rotation, in a first direction 122 between the ratchet gear member 101 and the ratchet pawl member 102. Relative motion, e.g. rotation, between the ratchet gear member 101 and the ratchet pawl member 102 is blocked in the opposite or second direction 120. Hence, a first state of the multistate ratchet mechanism 100 is obtained where the ratchet pawl member 101 is arranged at the first surface portion 110 of the ratchet gear member 101 with the ratchet finger 104 engaging a tooth of the first toothing.

The multi-state ratchet mechanism 100 is configured such that the ratchet pawl member 102 and the ratchet gear member 101 are relatively displaceable in an orthogonal direction to the previously discussed first and second directions 120, 122. This orthogonal direction may be an axial direction of an annular or cylindrical embodiment of the ratchet gear member as discussed below in further detail.

A second state of the multi-state ratchet mechanism 100 is obtained by displacing or moving the ratchet pawl member 102 to the second surface portion 113 of the ratchet gear member 101 such that the pawl finger 104 engages the oppositely oriented, relative to the first one-way toothing, second one-way toothing at the second surface portion 113.

FIG. 2 shows simplified schematic views 10, 20, 30, 40 of the multi-state ratchet mechanism 100 placed in this second state. In the second state of the multi-state ratchet mechanism 100, relative motion, e.g. rotation, between the ratchet gear member 100 and the ratchet pawl member 102 is blocked in the first direction 122 and enabled in the second direction 120 due to the opposite orientation of the second one-way toothing arranged on the second surface portion 113 of the ratchet gear member 101 relative to the orientation of the first one-way toothing described above. Each asymmetrical tooth of the second asymmetrical toothing comprises a first sloped edge 108a and a second sloped edge 108b wherein a slope of the first sloped edge 108a is steeper than a slope of the second sloped edge 108b as best illustrated on view 30 showing the cross-sectional profile of the first asymmetrical toothing. Hence, by displacing the ratchet pawl member 102 between these oppositely orientated one-way toothings of the first and second surface portions 110, 113 of the ratchet gear member 101, different rotationally locked states, or different movement locked states, of the multi-state ratchet mechanism 100 are provided in a relatively simple manner requiring few separate parts or components.

A third or intermediary state of the multi-state ratchet mechanism 100 is illustrated on FIG. 3. The intermediary state is obtained by displacing or moving the ratchet pawl member 102 to a third surface portion 117 of the ratchet gear member 101 which is a transitional surface arranged in-between the first and second surface portions 110, 113. FIG. 3 shows simplified schematic views 10, 20, 30, 40 of the multi-state ratchet mechanism 100 placed in this intermediary state. In this intermediary state, the pawl finger 104 engages the teeth of both the first one-way toothing at the first surface portion 110 and the teeth of the second one-way toothing at the second surface portion 113 due to the width of the pawl finger 104. More specifically, in the intermediary state the forward oriented stop surface 106 of the pawl finger 104 engages above-discussed first steep sloped edge 110a of a tooth of the first one-way toothing while the backward oriented stop surface 107 of the pawl finger 104 is configured to engage the corresponding steep sloped edge 108a of a tooth of the second, oppositely orientated, one-way toothing of the second surface portion 113 as best illustrated on the cross-sectional view 40 in conjunction with the perspective view 10. Finally, the third sloped edge 110b is situated in-between the first steep sloped edge 110a and the second steep sloped edge 108a. Hence, the cross-sectional profile of the toothing of the third surface portion 117 is unconventional with three sloped edges interacting with the pawl finger 104. The respective slopes of the first and second sloped edges 110a, 108a are markedly steeper than the slope of the third sloped edge 110b. The slope of the first sloped edge 110a may be substantially identical to the slope of the second slope edge 108a. Hence, in this intermediary state of the multi-state ratchet mechanism 100, relative motion, e.g. rotation, between the ratchet gear member 100 and the ratchet pawl member 102 is blocked in both the first and second directions 120, 122, respectively. Hence, by displacing the ratchet pawl member 102 between the first surface portion 110, the second portion 113 and the third/intermediary surface portion 117 of the ratchet gear member 101, three different rotationally locked states, or different movement locked states, of the multi-state ratchet mechanism 100 are provided in a relatively simple manner requiring few separate parts or components of the multi-state ratchet mechanism 100.

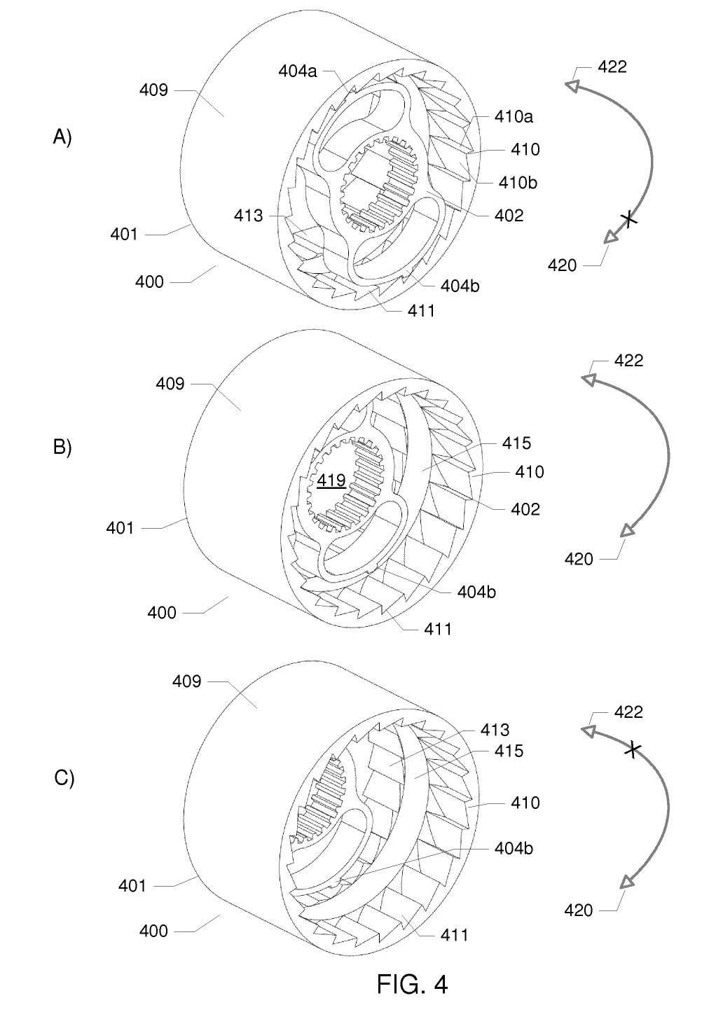

FIGS. 4A), B) and C) are respective schematic perspective views of a multi-state ratchet mechanism 400 in three different operational states thereof in accordance with a first embodiment of the invention. The multi-state ratchet mechanism 400 is suitable for use in a drug injection device or other types of injection devices. FIG. 4A) shows the multi-state ratchet mechanism 400 in a first state and FIGS. 4B) and C) show second and third further states of the multi-state ratchet mechanism 400 as discussed below.

The multi-state ratchet mechanism 400 comprises a cylindrical ratchet gear member 401 and a cooperating ratchet pawl member 402 as illustrated in the perspective view of FIG. 4A). A first finger or tooth 404a and a second finger or tooth 404b of the ratchet pawl member 402 are both biased against a first asymmetrical toothing 410 arranged on a first ring shaped surface portion 411 of an inner annular surface of the cylindrical ratchet gear member 401. In this manner the first and second fingers 404a, 404b engage respective teeth of the first asymmetrical toothing 410. The cylindrical ratchet gear member 401 further comprises a cylindrical outer surface 409 of predetermined dimensions which outer surface in practice may be a cylindrical wall section of a user operable button or a housing exterior of the drug injection device (not shown) in which the multi-state ratchet mechanism 400 is integrated. The cylindrical outer surface 409 may comprise corrugations to improve user grip, manipulation and actuation. The first asymmetrical toothing 410 of the first ring shaped surface portion 411 comprises a plurality of asymmetrical teeth having a first predetermined cross-sectional profile. Each asymmetrical tooth of the first asymmetrical or one-way toothing comprises a first sloped edge 410a and a second sloped edge 410b wherein a slope of the first sloped edge 410a is steeper than a slope of the second sloped edge 410b as illustrated on the drawing. The ratchet pawl member 402 is axially displaceable along a central axis (not shown) of the cylindrical ratchet gear member 401 between the first ring shaped surface portion 411 of the inner annular surface of the cylindrical ratchet gear member 401 and a second ring shaped surface portion 415 (refer to FIG. 4B)) and a third ring shaped surface portion 413 of the inner annular surface of the cylindrical ratchet gear member 401. When the ratchet pawl member 402 is positioned at the first ring shaped surface portion 411, the pawl fingers 404a, 404b are biased against the first asymmetrical toothing 410 such that forward and backward oriented stop surfaces of each pawl finger engage or contact a tooth in a similar manner as the one discussed above in connection with the multi-state ratchet mechanism 100 depicted on FIGS. 1-3. Consequently, the one-way or asymmetrical toothing 410 of the first ring shaped surface portion 411 of the cylindrical ratchet gear member 401 in cooperation with the pawl fingers 404a, 404b provides unidirectional rotation in a first direction 422 between the cylindrical ratchet gear member 401 and the ratchet pawl member 402. Relative rotational motion between the cylindrical ratchet gear member 401 and the ratchet pawl member 402 is blocked in the opposite direction 420. Hence, the first operational state of the multi-state ratchet mechanism 400 is obtained where the ratchet pawl member 401 is arranged at the first ring shaped surface portion 411 of the cylindrical ratchet gear member 401.

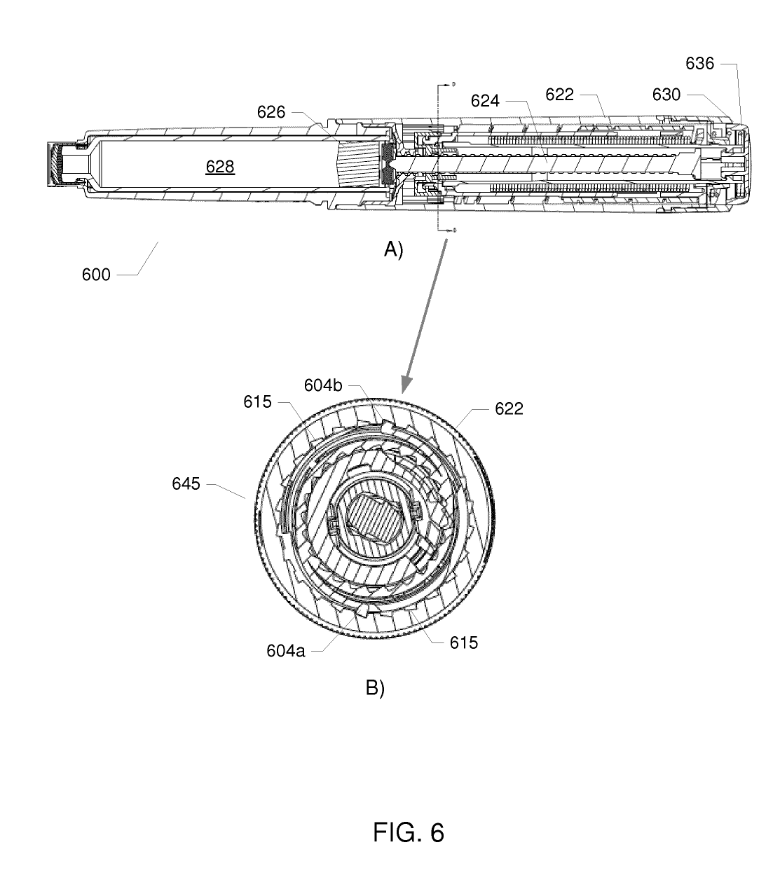

As illustrated in the perspective view of FIG. 4B), the inner annular surface of the cylindrical ratchet gear member 401 further comprises a second ring shaped surface portion 415 arranged at a second axial position of the inner annular surface adjacent to the first ring shaped surface portion 411 which comprises the above-discussed first asymmetrical toothing. The second ring shaped surface portion 415 comprises a substantially smooth surface that may be configured to physically engage the pawl fingers 404a, 404b to provide a certain amount of friction between the ratchet pawl member 402 and cylindrical ratchet gear member 401 during relative rotation between these members 401, 402. In an alternative embodiment, the substantially smooth surface 415 may be configured to lack physical contact with the pawl fingers 404a, 404b such that the ratchet pawl member 402 and cylindrical ratchet gear member 401 are freely rotatable substantially without any rotational friction. In yet another alternative embodiment, the second ring shaped surface portion 415 comprises a plurality of uniform sloped protrusions configured for engagement with the pawl fingers 404a, 404b to provide tactile feedback of a user of the multi-state ratchet mechanism 400 manipulating the cylindrical ratchet gear member 401 by gripping the outer surface thereof. Each of the plurality of uniform sloped protrusions may extend axially across the second ring shaped surface portion 415. The number of uniform sloped protrusions may for example correspond to the number of teeth of the one-way toothing 410 arranged on the adjacently arranged first ring shaped surface portion 411. The width of each of the pawl fingers 404a, 404b in the axial direction of the cylindrical ratchet gear member 401 is smaller than an axial width of the second ring shaped surface portion 415 such that the pawl fingers 404a, 404b do not engage any of the one-way toothings protruding from the first and third ring shaped surface portions 411, 413 surrounding the second ring shaped surface portion 415 on both sides. Hence, the skilled person will understand that each of these cross-sectional profiles of the second ring shaped surface portion 415 enables bi-directional relative rotation, i.e. rotation in both the first and second directions 422, 442, between the ratchet pawl member 402 and the cylindrical ratchet gear member 401 in the second state of the multi-state ratchet mechanism 400. The cylindrical ratchet gear member 401 comprises a central aperture 419 with a toothed surface mating to a threaded axially extending piston rod (not shown) of an injection structure of a drug injection device discussed below with reference to FIGS. 6, 7, 8 and 9. The central aperture 419 accordingly forms a coupling member to the injection structure.

As illustrated in the perspective view of FIG. 4C), the inner annular surface 411 of the cylindrical ratchet gear member 401 further comprises an, entirely optional, third ring shaped surface portion 413 arranged at a third axial position of the inner annular surface 411 and adjacent to the second ring shaped surface portion 415 discussed above. Hence, the second ring shaped surface portion 415 is placed in-between the first and third ring shaped surface portions in axial direction of the cylindrical ratchet gear member 401. The third ring shaped surface portion 413 comprises a second asymmetrical toothing comprising a plurality of asymmetrical teeth similar to the first asymmetrical toothing comprised on the first ring shaped surface portion 411. However, the teeth of the second asymmetrical toothing are oriented oppositely to the teeth of the first asymmetrical toothing 410 by a differently oriented cross-sectional profile. Each asymmetrical tooth of the second asymmetrical or one-way toothing may comprise a first sloped edge and a second sloped edge wherein a slope of the first sloped edge is steeper than a slope of the second sloped edge as discussed above in connection with the first asymmetrical toothing. However, the relative slopes of the first and second sloped edges of the teeth of the second asymmetrical toothing are reversed compared to the slopes of the teeth of the first asymmetrical toothing. In this manner, the second one-way toothing 420 formed on the third ring shaped surface portion 413 is oriented oppositely to the one-way toothing of the first ring shaped surface portion 411. Consequently, the one-way or asymmetrical toothing 420 of the third ring shaped surface portion of the cylindrical ratchet gear member 401 in cooperation with the pawl fingers 404a, 404b provides unidirectional rotation in a second direction 420 between the cylindrical ratchet gear member 401 and the ratchet pawl member 402. Relative rotational motion is on the other hand blocked in the first and opposite directions 422. Hence, the third operational state of the multi-state ratchet mechanism 400 is obtained where the ratchet pawl member 401 is arranged at the third ring shaped surface portion 440 of the cylindrical ratchet gear member 401.

The multi-state ratchet mechanism 400 is configured such that the ratchet pawl member 402 and the cylindrical ratchet gear member 401 are relatively displaceable in an axial direction of the cylindrical ratchet gear member 401.

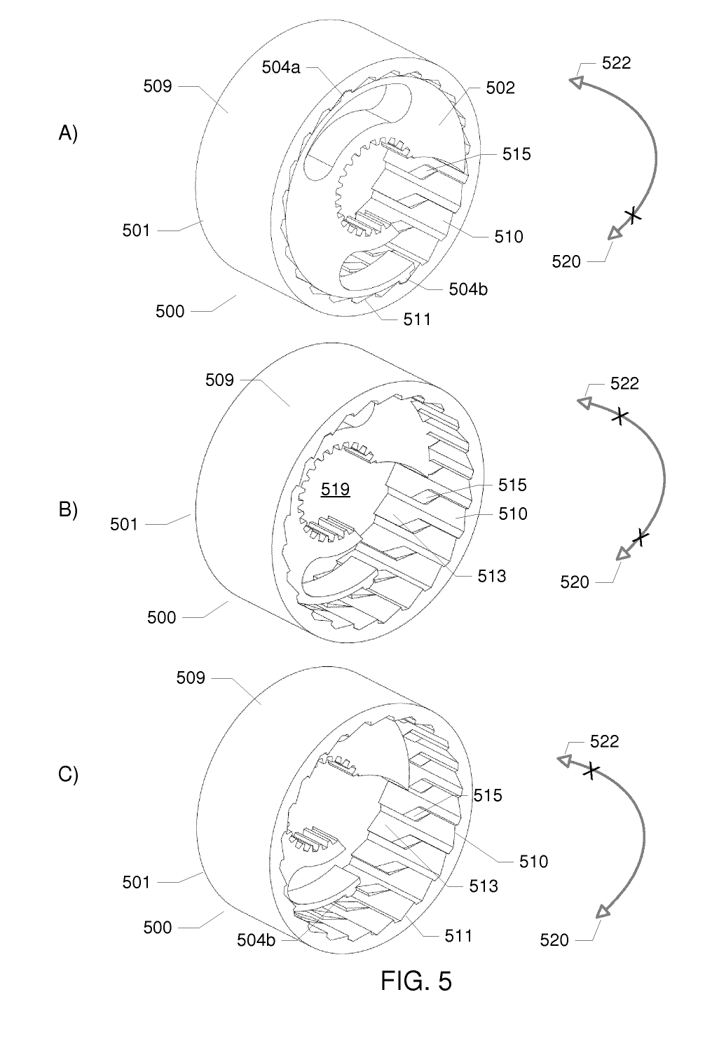

FIGS. 5A), B) and C) are respective schematic perspective views of a multi-state ratchet mechanism 500 in three different operational states thereof in accordance with a second embodiment of the invention. The multi-state ratchet mechanism 500 is suitable for use in a drug injection device or other types of injection devices. FIG. 5A) shows the multi-state ratchet mechanism 500 in a first state and FIGS. 5B) and C) show second and third further states of the multi-state ratchet mechanism 500 as discussed below. Corresponding features of the first and second embodiments of the multi-state ratchet mechanism 400, 500, respectively, have been provided with corresponding reference numerals to ease comparison.

The multi-state ratchet mechanism 500 comprises a cylindrical ratchet gear member 501 and a cooperating ratchet pawl member 502 as illustrated in the perspective view of FIG. 5A). A first asymmetrical toothing 510 is arranged on a first ring shaped surface portion 511 of an inner annular surface of the cylindrical ratchet gear member 501 similar to the first one-way or asymmetrical toothing 410 of the above-discussed first embodiment of the cylindrical ratchet gear member 401. As illustrated in the perspective views of FIGS. 5B) and 5C), the inner annular surface of the cylindrical ratchet gear member 501 further comprises an, entirely optional, third ring shaped surface portion 513 arranged at a third axial position, adjacent to a second axial position 515 discussed below in additional detail, of the inner annular surface of the cylindrical ratchet gear member 501. The third ring shaped surface portion 513 comprises a second asymmetrical toothing comprising a plurality of asymmetrical teeth that may be similar to the teeth of the first asymmetrical toothing 510 arranged on the first ring shaped surface portion 511 except for the opposite orientation of the second asymmetrical toothing. Hence, the position and shapes of the oppositely oriented first and second asymmetrical toothing of the cylindrical ratchet gear member 501 may be substantially identical to the position and shapes of the oppositely oriented first and second asymmetrical toothing of the first embodiment of the ratchet gear member 401 discussed above. The perspective view of FIG. 5C) shows the ratchet pawl member 502 arranged at the third ring shaped surface portion 513 of the cylindrical ratchet gear member 501. The cylindrical ratchet gear member 501 comprises a central aperture 519 with a toothed surface mating to a threaded axially extending piston rod (not shown) of an injection structure of the drug injection device discussed below with reference to FIGS. 6, 7, 8 and 9. The central aperture 519 accordingly forms a coupling member to the injection structure.

However, the cylindrical ratchet gear member 501 differs from the corresponding cylindrical ratchet gear member 401 according to the first embodiment by the cross-sectional profile of the second ring shaped surface portion 515 of the inner surface. This second ring shaped surface portion 515 is placed in-between the first and third ring shaped surface portions 511, 513, respectively, in axial direction of the cylindrical ratchet gear member 501. The perspective view of FIG. 5B) shows the ratchet pawl member 502 arranged at the second ring shaped surface portion 515 of the cylindrical ratchet gear member 501. A toothing of the second ring shaped surface portion 515 comprises an unconventional cross-sectional profile with at least three sloped edges such that each tooth is capable of locking both a forward oriented stop surface (not shown) of the pawl finger 504b and a backward oriented stop surface (not shown) of the pawl finger 504b when the pawl finger 504b engages the toothing of the second ring shaped surface portion 515 similarly to the engagement and bi-directional locking of the pawl finger 104 when placed on the intermediary surface portion 117 of the exemplary ratchet gear member 101 discussed above in connection with FIG. 3. The forward and backward oriented stop surfaces of the pawl finger 504b may be similar to the forward and backward oriented stop surfaces 106, 107, respectively, of the exemplary pawl finger 104 illustrated on FIG. 1. This unconventional cross-sectional profile of the toothing of the third ring shaped surface portion 515 may largely correspond to the cross-sectional profile of the intermediary surface portion 117 of the exemplary ratchet gear member 101 discussed above in connection with FIG. 3. Hence, the cross-sectional profile of the toothing of the third ring shaped surface portion 515 may comprise a first sloped edge and a second sloped edge wherein a slope of the first sloped edge is steeper than a slope of the second sloped edge; and a third sloped edge arranged in-between the first sloped edge and the second sloped edge, wherein a slope of the third sloped edge is steeper than the slope of the second sloped edge. The first sloped edge is configured for engaging the forward facing radially projecting stop surfaces of the pawl tooth 504b and the third sloped edge is configured for engaging the backwards facing radially projecting stop surface of the pawl tooth 504b. Consequently, the unconventional cross-sectional profile of each tooth of the toothing of the second ring shaped surface portion 515 provides bi-directional rotational locking, i.e. rotational locking in both the first and second directions 520, 522, between the ratchet pawl member 502 and cylindrical ratchet gear member 501 in a second state of the multi-state ratchet mechanism 500. Hence, the multi-state ratchet mechanism 500 comprises three different operational states where the first state provides unidirectional rotation in the first direction 522, the third state allows unidirectional rotation in the second, opposite, direction 520 and the second state provides bi-directional rotational locking between the cylindrical ratchet gear member 501 and the ratchet pawl member 502. Each of these three distinct states of the multi-state ratchet mechanism 500 can be selected or set by choosing the appropriate relative axial displacement between the ratchet pawl member 502 and the cylindrical ratchet gear member 501.

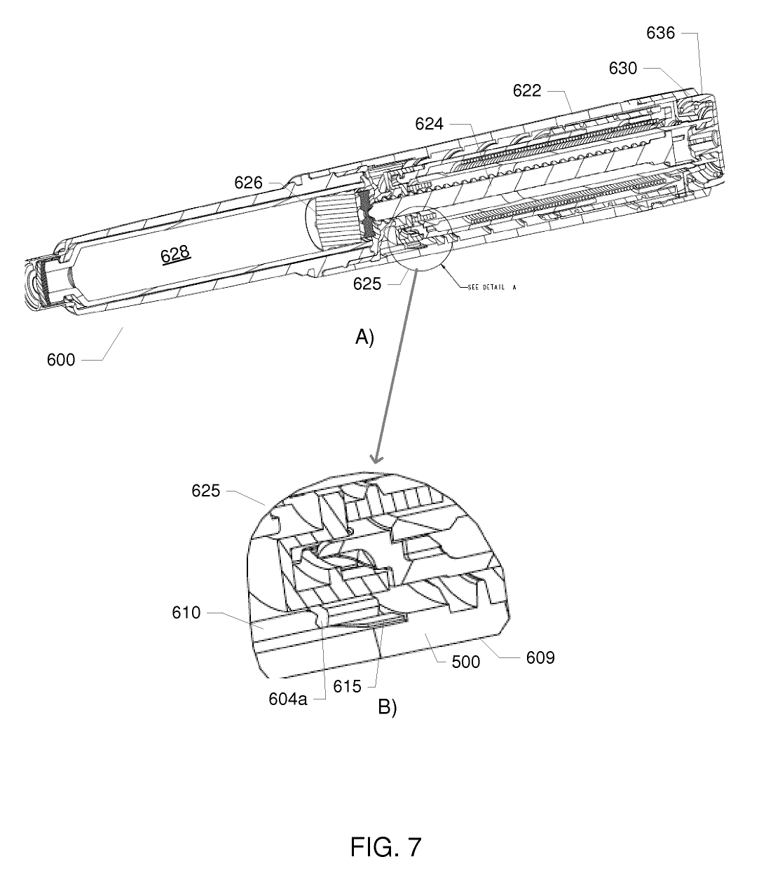

FIG. 6A) shows an axial cross-sectional view of a drug injection device 600 comprising a multi-state ratchet mechanism according to any of the first and second embodiments thereof discussed above. The drug injection device 600 is placed in an inactive state as discussed below in additional detail. FIG. 6B) shows a radial cross-sectional view of the drug injection device 600 taken through the multi-state ratchet mechanism along indicated line D-D. The drug injection device 600 comprises a hollowing elongate housing structure 622 and an injection structure arranged inside the hollow housing structure 622. The injection structure comprises a threaded axially extending piston rod 624 coupled to an axially displaceable or movable piston 626. The threaded axially extending piston rod 624 is configured to advance the movable piston 626 a predetermined axial distance inside a drug cartridge chamber 628 to deliver or inject a predetermined dose of drug to a patient or user. The drug injection device 600 further comprises a user operable dose dial 630 coupled to a dose adjustment structure. The drug injection device 600 comprises an axially displaceable user actuable button 636 coupled to the cylindrical ratchet gear member (500) (refer to the expanded partial view 625 of FIG. 7B)). In the radial cross-sectional view 645 of the drug injection device 600 a first finger 604a and a second finger 604b of a ratchet pawl member (not shown) are biased against respective asymmetrical teeth of a first toothing 615 protruding from a first inner ring shaped surface portion of a ratchet gear member.

FIG. 7A) shows a second axial cross-sectional view of the drug injection device 600 comprising the multi-state ratchet mechanism according to any of the first and second embodiments thereof discussed above. The skilled person will understand that the ratchet gear member (500) is integrally formed with the housing structure 622 of the drug injection device 600 such that the outer cylindrical surface 609 of the ratchet gear member forms a section of the housing surface of the housing 622. As illustrated in the expanded partial view 625 of FIG. 7B), a first toothing 615 with the previously discussed unconventional cross-sectional profile projects from the first inner ring shaped surface portion of the ratchet gear member (500). As discussed above, the first finger 604a of the ratchet pawl member (not shown) is biased against an asymmetrical tooth of the first toothing 615. Hence, the first toothing 615 of this first inner ring shaped surface portion ensures bi-directional rotational locking between the ratchet pawl member and the ratchet gear member 500 in an inactive state of the drug injection device 600 such that the ratchet pawl member is locked to the housing 622 of the drug injection device 600. In this inactive state, the user operable dose dial 630 may be rotated and compress a compression spring such that a desired drug dose to be administered is set or selected.

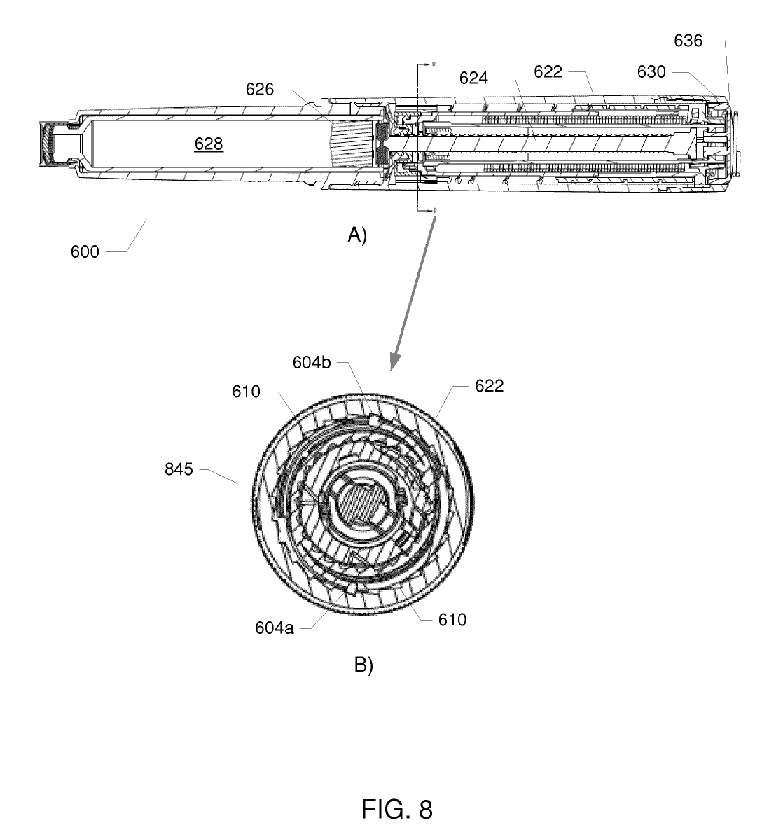

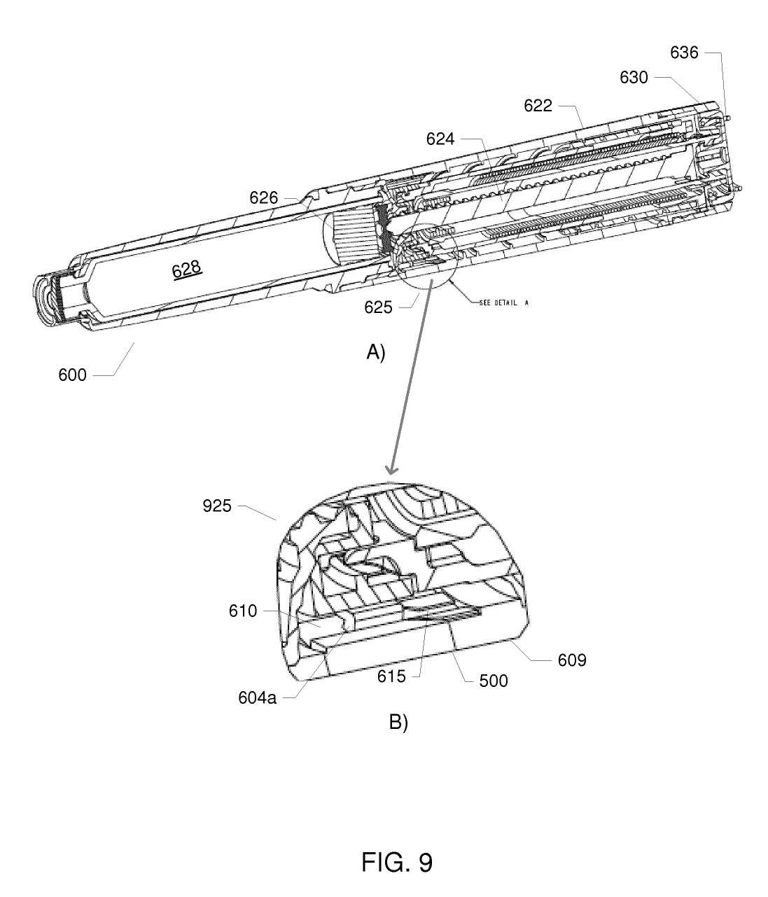

FIG. 8A) shows a third axial cross-sectional view of the drug injection device 600 discussed above. The drug injection device 600 is placed in an active state or dosing state as discussed below in additional detail. FIG. 8B) shows a radial cross-sectional view of the drug injection device 600 in the active state taken through the multi-state ratchet mechanism along indicated line D-D. The axially displaceable user actuable button 636 has been depressed pushing the ratchet gear member (500) (refer to the expanded partial view 925 of FIG. 9B)). In the radial cross-sectional view 845 of the drug injection device 600, the first finger 604a and the second finger 604b of the ratchet pawl member (not shown) are biased against respective asymmetrical teeth of the above-discussed second, one-way, toothing 610 protruding from the second inner ring shaped surface portion of a ratchet gear member.

FIG. 9A) shows a fourth axial cross-sectional view of the drug injection device 600 discussed above. The drug injection device 600 is placed in the active state or dosing state. As illustrated in the expanded partial view 925 of FIG. 9B), the first toothing 615 with the previously discussed unconventional cross-sectional profile projects from the first inner ring shaped surface portion of the ratchet gear member (500). However, in this active state of the drug injection device the first finger 604a of the ratchet pawl member (not shown) is biased against a one-way or asymmetrical tooth of the second toothing 610 rather than against the first toothing as in the inactive state. This shift of toothing from the first to the second toothing is caused by the depression of the axially displaceable user actuable button 636 which has axially displaced the ratchet pawl member. Hence, the first finger 604a rests on the second toothing 610 of the ratchet gear member, which toothing 610 is a conventional one-way toothing. The latter property ensures unidirectional rotational locking between the ratchet pawl member and the ratchet gear member 500 in the depicted active state of the drug injection device 600 such that the ratchet pawl member is locked to the housing 622 of the drug injection device 600 in only one rotational direction. In this active state, the force build-up in the compression spring may be released to push the axially displaceable piston 626 in a distal direction and expel the selected dose of drug from the drug cartridge chamber 628.

1. A multi-state ratchet mechanism for a drug injection device, comprising:
a ratchet gear member comprising an outer surface of predetermined shape and dimensions and an inner annular surface, wherein the inner annular surface comprises:
a first ring shaped surface portion with a first predetermined cross-sectional profile comprising a first asymmetrical toothing arranged at a first axial position of the inner annular surface,
each asymmetrical tooth of the first asymmetrical toothing comprising a first sloped edge and a second sloped edge wherein a slope of the first sloped edge is steeper than a slope of the second sloped edge;
a second ring shaped surface portion arranged at a second axial position of the inner surface axially spaced apart from the first axial position and having a second predetermined cross-sectional profile differing from the first predetermined cross-sectional profile;
a ratchet pawl member axially displaceable between the first ring shaped surface portion and the second ring shaped surface portion to provide first and second operating states, respectively, of the multi-state ratchet mechanism, the ratchet pawl member comprising:
a pawl tooth or finger comprising a forward facing radially projecting stop surface configured for engagement with the first sloped edge or the second sloped edge of each asymmetrical tooth and a backward facing radially projecting stop surface configured for engagement with the opposite one of the first and second sloped edges of each asymmetrical tooth to, in the first state of the multi-state ratchet mechanism, provide unidirectional rotation in a first direction between the ratchet gear member and the ratchet pawl member.

2. A multi-state ratchet mechanism according to claim 1, wherein the second predetermined cross-sectional profile of the second ring shaped surface portion of the ratchet gear member comprises:
a substantially smooth surface physically engaging the pawl tooth or finger to, in the second state, provide bi-directional rotation between the ratchet gear member and the ratchet pawl member; or
a plurality of uniform sloped protrusions configured for engagement with the pawl tooth or finger to, in the second state, provide bi-directional rotation between the ratchet gear member and the ratchet pawl member.

3. A multi-state ratchet mechanism according to claim 1, wherein the second predetermined cross-sectional profile of the second ring shaped surface portion comprises a second asymmetrical toothing, orientated oppositely to the first asymmetrical toothing,
each asymmetrical tooth of the second asymmetrical toothing comprising a first sloped edge and a second sloped edge wherein a slope of the first sloped edge is steeper than a slope of the second sloped edge to, in the second state of the multi-state ratchet mechanism, provide unidirectional rotation in a second direction, oppositely to the first direction, between the ratchet gear member and the ratchet pawl member in the second state of the multi-state ratchet mechanism.

4. A multi-state ratchet mechanism according to claim 1, wherein the second predetermined cross-sectional profile of the second ring shaped surface portion comprises:
a first sloped edge and a second sloped edge wherein a slope of the first sloped edge is steeper than a slope of the second sloped edge; and
a third sloped edge arranged in-between the first sloped edge and the second sloped edge, wherein a slope of the third sloped edge is steeper than the slope of the second sloped edge,
such that the first sloped edge is configured for engaging the forward facing radially projecting stop surfaces of the pawl tooth and the third sloped edge is configured for engaging the backwards facing radially projecting stop surface of the pawl tooth thereby providing bidirectional rotational locking between the ratchet gear member and the ratchet pawl member in the second state of the multi-state ratchet mechanism.

5. A multi-state ratchet mechanism according to claim 4, wherein the slope of the first sloped edge is substantially identical to the slope of the third sloped edge in the second predetermined cross-sectional profile of the second ring shaped surface portion.

6. A multi-state ratchet mechanism according to claim 1, wherein the inner annular surface of the ratchet gear member comprises a third ring shaped surface portion having a third predetermined cross-sectional profile,
wherein the third ring shaped surface portion is arranged at a third axial position of the inner annular surface,
wherein the third predetermined cross-sectional profile differs from each of the first and second predetermined cross-sectional profiles; the ratchet pawl member being axially displaceable to the third ring shaped surface portion in a third state of the multi-state ratchet mechanism.

7. A multi-state ratchet mechanism according to claim 6, wherein the third ring shaped surface portion is arranged in-between the first and second ring shaped surface portions of the ratchet gear member in the axial direction of the ratchet gear member.

8. A multi-state ratchet mechanism according to claim 1, wherein the ratchet pawl member comprises a substantially flat annular body portion having a substantially ring shaped outer surface and a central through going teethed aperture for receipt of a mating member such as a mating piston rod.

9. A multi-state ratchet mechanism according to claim 8, wherein the substantially flat annular body portion of the ratchet pawl member comprises an elongate curved through going aperture extending along a first portion of the substantially ring shaped outer surface to form a narrow curved wall segment; wherein the narrow curved wall segment comprises the pawl finger.

10. A multi-state ratchet mechanism according to claim 1, wherein the ratchet pawl member comprises a second tooth or pawl finger with substantially identical shape and dimensions to the pawl tooth or finger.

11. A multi-state ratchet mechanism according to claim 1, wherein the outer surface of the ratchet gear member has a cylindrical shape with an outer diameter less than 30 mm.

12. A drug injection device comprising:
a hollowing elongate housing structure,
an injection structure arranged inside the hollow housing structure and comprising a threaded axially extending piston rod coupled to a movable piston,
wherein the threaded axially extending piston rod is configured to advance the piston a predetermined axial distance inside a drug cartridge to deliver a predetermined dose of drug,
a user operable dose dial coupled to a dose adjustment structure,
a multi-state ratchet mechanism according to any of the preceding claims wherein the ratchet pawl member is coupled to the threaded axially extending piston rod via a coupling member or structure,
an axially displaceable user actuatable button coupled to the ratchet gear member for axially displacing the ratchet gear member relative to the ratchet pawl member between at least the first and second states of the multi-state ratchet mechanism.
